(19) United States
(12) Patent Application Publication
(10) Pub. No.: US 2002/0155136 A1
(43) Pub. Date: Oct. 24, 2002

(54) MEDICINE CONTAINING YOHIMBINE AND ARGININE FOR TREATING ERECTILE DYSFUNCTION

(76) Inventor: PHILIPPE GORNY, PARIS (FR)

Correspondence Address: Edward D. Grieff, 1455 Pennsylvania Ave, N.W, Washington, DC, 20004-1008, US

(21) Appl. No.: 09/462,143

(22) PCT Filed: Jul. 6, 1998
(86) PCT No.: PCT/FR98/01438

(30) Foreign Application Priority Data
Jul. 4, 1997 (FR) 97 08504

(51) Int. Cl. A61K 9/00; A61K 35/78; A01N 65/00

(52) U.S. Cl. 424/400; 424/725

(57) ABSTRACT

The invention concerns the use in combination of yohimbine and arginine as active ingredients for preparing a medicine for treating erectile dysfunction. The combination has a marked synergistic effect.

The invention relates to the use, in combination, of yohimbine and arginine in the preparation of a medicinal product intended for treating erectile dysfunction.

It is known that the erection process is schematically that indicated below. The erectile tissue of the penis, known as the cavernous body, is a spongy tissue capable of filling with blood. At rest, the arteries of the penis are under the dependence of the adrenergic tonus which keeps them in spasmic form, such that no blood flow fills the cavernous body. In the event of appropriate stimulation, the nervi erigentes inhibit the adrenergic tonus, the arteries of the penis dilate and the cavernous body fills with blood, enlarges and the increase in the internal pressure makes it become rigid. By enlarging, it crushes the veins of the penis, preventing evacuation of the blood it contains, this ensuring that the rigidity is maintained. After ejaculation, adrenaline is again released locally, the arterial blood supply reduces immediately, the pressure in the cavernous body decreases and the blood accumulated therein can be evacuated by the veins which are no longer compressed, thus resulting in the loss of rigidity, with return to the resting state.

It is known that a relatively large proportion of men suffer from permanent or temporary erectile dysfunction. These disorders can be of organic origin, in which case they require specific treatments adapted to each case. However, a majority of non-organic erectile dysfunctions are observed, often of psychogenic origin. For these latter cases, various treatments are available. Intra-cavernous injection of vasoactive substances is capable of providing good results, but is not readily accepted by a large proportion of patients. Oral-route treatments are generally better accepted. To this end, various products have been proposed, often of plant origin: ginseng, ginger, yohimbine, cola nut, spiruline, ylang-ylang, hogweed, cinnamon, etc.

Yohimbine is a substance extracted from the bark of the plant Corynanthe yohimbe. It has antagonistic properties towards the presynaptic alpha-2-adrenergic receptors. In other words, it inhibits the effects of adrenaline and thus promotes irrigation of the cavernous body by the arterial blood. It has been proposed in the treatment of impotence of psychogenic origin. Certain studies published on this subject reveal improvements in about 35 to 45% of cases with yohimbine, but these results are controversial. In addition, various side effects such as vertigo, anxiety, nervousness, headaches, insomnia and an increase in arterial pressure have been observed, although for relatively large doses; see for example The Medical Letter, French Edition, Vol. 17, No. 2, 5-6 (ML USA No. 938), 1995.

The administration of L-arginine has also been proposed. The administration of arginine is thought to be manifested in an effect on muscle relaxation of the arteries and of the cavernous body, this relaxation being necessary to obtain an erection. The administration of 2800 mg per day of L-arg-nine is said to have a favourable effect on erectile dysfunction in approximately 40% of cases; see A. W. Zorgniotti and E. F. Lizza, Int. J. Impotence Res., 6, 33-36 (1994).

It has now been discovered that, in this field, the combination of yohimbine and arginine has advantageous properties. Specifically, tests have shown that the combination of arginine and yohimbine makes it possible to obtain favourable results in the treatment of erectile dysfunction, mainly non-organic dysfunction, using lower doses of yohimbine than those used previously, by virtue of a synergistic effect.

With the combination of yohimbine and arginine, it is also possible to reduce the doses of arginine used.

In many cases, this combination appears to act quite rapidly and can thus be used in the treatment of temporary erectile dysfunction.

One subject of the invention is thus the use, in combination, of yohimbine and arginine as active ingredients in the preparation of a medicinal product intended for treating non-organic erectile dysfunction.

Yohimbine and arginine can be used in the form of the free base or in the form of a pharmaceutically acceptable salt.

The active ingredients in the medicinal product of the invention can be presented separately, each in a suitable pharmaceutical form, and combined in the same packaging.

However, to facilitate the simultaneous administration of the active ingredients, it is generally preferred to prepare the medicinal product of the invention in a pharmaceutical form containing both yohimbine and arginine.

The medicinal product of the invention can be prepared in a pharmaceutical form which allows the administration of a sufficient dose of yohimbine, in particular a dose of from 2 to 8 mg, and in particular from 2 to 6 mg per day, taken as one or two doses. This dose is calculated as the weight of yohimbine in the form of the free base.

The medicinal product of the invention is prepared in a pharmaceutical form which allows the administration of a sufficient dose of arginine, which is, for example, a dose of from 1 to 4 g per day, and in particular from 1 to 2 g per day, taken in one or two doses, the said dose being calculated as the weight of arginine in the form of the free base.

The medicinal product of the invention can be administered orally, sublingually, nasally, rectally or cutaneously.

To this end, it can be presented in any form which allows oral administration (in particular in the form of gel capsules, drinkable solutions, granules, lozenges or tablets), nasal administration (solutions to be administered in the form of drops or sprays), rectal administration (suppositories) or cutaneous administration (ointments or patches).

These pharmaceutical forms are prepared in the usual way and can contain suitable standard excipients and vehicles.

Yohimbine can be used in the form of the free base or in the form of a salt such as the hydrochloride.

Arginine can be used in the form of the free base or of a pharmaceutically acceptable salt, such as the hydrochloride, glutamate, aspartate or citrate.

The duration of the treatment can range, for example, from 2 to 4 weeks or more. An episodic use can also be envisaged.

The examples which follow illustrate the invention.

### EXAMPLE 1

A gel capsule was prepared, consisting of a gelatine capsule containing:

arginine: 0.5 g

yohimbine: 1 mg

### EXAMPLE 2

Randomized double-blind tests were carried out on 42 adult married men aged from 28 to 64 years, suffering from erectile dysfunction with no detectable organic cause.

The treatment consisted in administering either (in 21 cases) 4 gel capsules as described in Example 1 above (2 in the morning and 2 in the evening) or (in 21 cases), in a similar manner, a placebo (4 gel capsules of identical appearance but containing only 0.5 g of lactose).

The individuals on whom the test was carried out, and likewise the individuals supplying the test compounds, were unaware as to whether they received or supplied the yohimbine+arginine combination or the placebo.

After 2 weeks, the individuals undergoing the test were questioned, by asking them if they had observed an improvement in erectile function.

Analysis of the results made it possible to draw the following conclusions:

in the individuals suffering from transient erectile dysfunction (22 cases), 12 received the placebo. 7 cases of improvement with the combination studied and 4 cases of improvement with the placebo were found;

in the individuals suffering from chronic erectile dysfunction (20 cases), 9 received the placebo. 4 cases of improvement with the combination tested and 1 case of improvement with the placebo were found.

1. Use, in combination, of yohimbine and arginine as active ingredients in the preparation of a medicinal product intended for treating erectile dysfunction.

2. Use according to claim 1, in which the yohimbine and/or arginine are used in the form of the free base or in the form of a salt.

3. Use according to claim 1, in which the medicinal product is prepared in a pharmaceutical form which allows simultaneous administration of the active ingredients.

4. Use according to any one of the preceding claims, characterized in that the said medicinal product is prepared in a pharmaceutical form which allows the administration of a dose of from 2 to 8 mg of yohimbine, taken in one or two doses, the said dose being calculated as the weight of yohimbine in the form of the free base.

5. Use according to any one of the preceding claims, characterized in that the said medicinal product is prepared in a pharmaceutical form which allows the administration of a dose of from 1 to 4 g, and in particular from 1 to 2 g, of arginine, taken in one or two doses, the said dose being calculated as the weight of arginine in the form of the free base.

6. Medicinal product intended for treating erectile dysfunction, characterized in that it contains as active ingredients arginine, in free or salified form, and yohimbine, in free or salified form.

7. Medicinal product according to the preceding claim, characterized in that it contains the said active ingredients separately, in the same packaging.

8. Medicinal product according to claim 6, characterized in that it is in a single pharmaceutical form containing the two active ingredients.

9. Medicinal product according to any one of claims 6 to 8, characterized in that it is in a pharmaceutical form which allows the administration of a dose of from 2 to 8 mg, and in particular from 2 to 6 mg, of yohimbine, taken in one or two doses, the said dose being calculated as the weight of yohimbine in the form of the free base.

10. Medicinal product according to any one of claims 6 to 9, characterized in that it is in a pharmaceutical form which allows the administration of a dose of from 1 to 4 g, and in particular from 1 to 2 g, of arginine, taken in one or two doses, the said dose being calculated as the weight of arginine in the form of the free base.

11. Medicinal product according to any one of claims 6 to 10, characterized in that it is in the form of gel capsules, drinkable solutions, granules, lozenges, tablets, ointments, patches, suppositories or nasal solutions to be administered in the form of drops or sprays.
